(19) United States
(12) Patent Application Publication
(10) Pub. No.: US 2003/0114403 A1
(43) Pub. Date: Jun. 19, 2003

(54) Method for treatment of cancers and antineoplastic pharmaceutical formulations

(75) Inventors: Adrian Mihail, Kokomo, IN (US); Nicolae Mihail, Cluj-Napoca (RO)

Correspondence Address: Adrian MIHAIL, 2515 S 400 E, KOKOMO, IN, 46902, US

(73) Assignee: CANADA Inc.

(21) Appl. No.: 10/025,074

(22) Filed: Dec. 19, 2001

(51) Int. Cl. A61K 48/00; A61K 31/655; A61K 31/19; A61K 31/137

(52) U.S. Cl. 514/44; 514/150; 514/648; 514/557

(57) ABSTRACT

A method of therapy based on a pharmaceutical composition that has an irreversible effect of inhibiting the malignancy of tumors and cancers that comprises an effective ingredient is disclosed. The effective ingredient can be used in combination with a vector and/or metabolite that is specific to the malignant cells. This effect of blocking the malignancy without killing the cells is new, specific, and has been called malignodepriving inactivation.

### REFERENCES:

1. Goldie, J. H., Coldman, A. J., Drug resistance in Cancer, Mechanisms and Models, Cambridge University Press, UK, 1998.

2. Dorr, R. T., Von Hoff, D. D., Cancer Chemotherapy Handbook, Appleton and Lange, Norwalk, Conn., 1994.

3. Busch M. The Molecular Biology of Cancer, Acad. Press, New York, 1974.

4. Kay D. Techniques for Electron Microscopy, Blackwell Sci. Pub. Oxford and Edinburgh, UK, 1967.

5. Lapis K., Bendeczki I. Ultrastructural Alterations caused by cytostatic sugar-alcohol derivatives, Cancer Research. V. 28, pp. 1256-1274, 1968.

6. Weakley S. B., A Beginner's Handbook in Biological Transmission Electron Microscopy, Churchill Livingstone, Edinburgh, London-Melbourne-New York, 1981.

### BACKGROUND OF THE INVENTION

This invention pertains to the field of cancer therapy. The three major challenges faced by medicine in the fight against the malignant neoplasm are: 1. To stop the uncontrollable cell proliferation resulting in unregulated growth of malignant tissue based on the genetic instability. 2. To suppress the ability of malignant cells to invade local and remote tissues (metastasis). 3. To prevent the malignant cells from developing drug resistance.

There is a lack of cell differentiation that is typical to malignant tissue and a lack of detectable symptoms to determine the incipience of erratic cell multiplication that characterizes cancer. That contributes to an increased difficulty of early stage detection of the neoplastic state.

There are many types of antineoplastic agents that have different degrees of effectiveness against cancers and tumor cells. Some types of cancers and tumors respond to these agents others do not. Most of the chemotherapeutic agents also destroy normal cells and are accompanied by adverse reactions.

In the ideal situation the antineoplastic agents would have specificity for cancer and tumor cells while not affecting normal cells. The development of drugs to target tumor cells only due a unique specificity has been the subject of research for decades. To date, no such drugs are known or have been found. Cytotoxic agents in use are focused on targeting cells that are dividing rapidly and those cells can be malignant or normal.

It is the object of this invention to provide a method of therapy that is based on a pharmaceutical anticancer composition where the active agent is only delivered to cancer cells. The mechanism is based on the tinctorial affinity of the malignant cells to the active agent. The active agent used in combination with a vector and/or metabolite that is specific to the cancer cell makes the targeting of the neoplastic cells even more efficient.

The active ingredient is selected from a group of chemicals called triphenylmethane dyes that are biological stains known to have antiseptic, antibacterial and antifungal properties. In one of the embodiments of the present invention the active ingredient is isometamidium chloride that is known as an antitrypanosomal agent used in veterinary medicine.

### BRIEF SUMMARY OF THE INVENTION

The object of this invention is to provide a method of cancer therapy. It is based on a pharmaceutical composition that produces an irreversible blockage of cancer, leukemia and tumors. The composition contains either a triphenylmethane dye or isometamidium chloride as the active anticancer agent. The composition can be used in combination with a specific vector and metabolite of the malignant cell.

The main advantages of this method of therapy are:

1. The type of action on neoplastic cells is by inhibition of signal transmission and reception. This results from the increase of the nuclear inter-membrane distance.

2. There are no side effects following the treatment.

3. For the effective concentrations of the compositions described in this invention there are no harmful effects on animals or humans.

4. The healthy tissues surrounding the tumors are not affected by the treatment as described by the present invention.

The above statements are addressed in detail by this specification.

### DETAILED DESCRIPTION OF THE INVENTION

A method for treatment of cancers in warm-blooded animals and humans, based on a pharmaceutical composition comprising a safe and effective amount of an antineoplastic compound. The antineoplastic compound is based on an active ingredient that is either selected from the group of chemicals known as triphenylmethane dyes or a chemical known as isometamidium chloride.

These compositions can be used to inhibit the growth and proliferation of cancers and other tumors in humans or animals by administration of an effective amount orally, rectally, topically, parenterally, intravenously or by direct injection into the tumor or any other route of administration. These compositions are used in concentrations that do not affect healthy cells.

A. Definitions

As used herein, “cancer” refers to all types of cancers, neoplasms, malignant tumors, leukemias and others that may be found in animals or humans.

As used herein, “pharmaceutical formulation” has a similar meaning with “anticancer compound” or “pharmaceutical composition” or “therapeutic compound” and are antineoplastic formulations of compounds that have the ability to prevent the proliferation of malignant cells. The active components of the antineoplastic formulations in this invention are the triphenylmethane dyes, their salts and isometamidium chloride. They are all described in this invention.

As used herein, “nuclear matrix” is the internal skeleton of the cell nucleus that can be defined as the structure that remains after removal of the cellular DNA and protein mass from the nucleus.

B. Dosage

Any suitable dosage may be given in the method of the invention. The amount will vary widely depending on the type of disease being treated (carcinoma, melanoma, sarcoma, leukemia etc.), the antineoplastic compound used, the species to be treated, body weight, tumor size, and type being treated.

Generally a dosage of between about 2 milligrams (mg) per kilogram (kg) of body weight and about 20 mg per kg of body weight is suitable, but this patent does not limit the dosage to these specific values. Preferably from 4 mg to about 15 mg/kg of body weight is used.

A dosage unit may comprise the components described in the examples of the application of this invention. The dosage unit can also comprise diluents, extenders and the like. The unit may be in gel, liquid or solid form suitable for oral, rectal, topical, intravenous injection or parenteral administration or injection into the tumor.

C. Delivery Forms

The active antineoplastic agents are typically mixed with a pharmaceutical carrier. This carrier can be a solid or liquid and the type is generally chosen based on the type of administration being used. A typical example that constitutes one of the embodiments of the present invention is when the active agent is administered in a liquid form. In this example the carrier is a suitable solvent vehicle. Pharmaceutically acceptable solvents are well known in the art. The most preferred solvent, due to its lack of toxicity and ease of delivery is water. There are other dosage forms that include pharmaceutically acceptable fats and oils, alcohols or other organic solvents, including esters, emulsions, or syrups. Liquid dosage forms may contain, for example, preservatives, suspending agents, sweeteners and thickeners that are all well known in the art.

There are numerous options in choosing the suitable carriers including lactose, sucrose, and gelatin. Oral dosage forms can contain flavorants. Parenteral and intravenous forms would also include minerals and other materials for compatibility with the type of injection or delivery system chosen.

Specific examples of pharmaceutically acceptable carriers and excipients that may be used to formulate the dosage forms of the present invention are described in referenced literature and so are the techniques for making dosage forms of the present invention.

D. Method of Treatment

The method of treatment can be any suitable method effective in the treatment of the particular cancer type that is being treated. Treatment may be oral, rectal, topical, parenteral or intravenous administration or by direct injection into the tumor. The method of applying an effective amount also varies depending on the tumor being treated. It is believed that intravenous, subcutaneous, or intramuscular application of the compounds, formulated with an appropriate carrier, will be the preferred method of administering the anticancer compounds to mammals and birds.

The countless experiments that have been performed show that the compositions described are effective in the inactivation of tumor cells without affecting healthy cells and therefore likely to be safe for use in animals and/or humans.

The examples that follow are illustrative; however it is understood that the present invention is not limited to such examples.

### DETAILED DESCRIPTION OF THE INVENTION BY EXAMPLES

A. Introduction

Malachite green and brilliant green, both triphenylmethane dyes, with lactic acid as specific vector and metabolite, produce an irreversible effect of blocking malignancies both in vitro and in vivo. In the following examples, lactic acid has been used as a specific vector to target malignant cells. However, other vectors may be used to increase the efficacy of the treatments. In the following cases, lactic acid is metabolized by the malignant cells and therefore potentiates the effect of the active anticancer compound (in the following examples triphenylmethane dyes).

This effect of blocking the malignancy without killing the cells is new, specific and has been called malignodepriving inactivation effect. It is a cancer inactivation that is not based on the known cytotoxic actions of chemotherapeutic agents in use. For those skilled in the art, the examination of treated cells under transmission electron microscopy (TEM) shows that the ultrastructural modifications that occur in the nucleus and cytoplasm of the treated cells are typical of this effect (See section D of this chapter). These modifications are followed by a rapid senescence of the cells. Chemically the explanation of the malignodepriving inactivation is believed to be an ester type reaction of the triphenylmethane dyes and the phosphorous free radicals of the nucleic acids.

From the cluster of diseases collectively known as cancer the following have been investigated with the use of triphenylmethane dyes: Jensen sarcoma, Ehrlich ascites, Guerin ascites, lymphoid leukemia and Marek's disease. Similar results were observed and documented in all cases. Therefore following is a summary of experiments performed with Ehrlich ascites.

Normal A2G mice were transplanted 24 hours prior to treatment with 1,000,000 Ehrlich ascites cells. Treatment procedures consisted of a single intraperitoneal injection of malachite green 4/mg/kg or brilliant green 2 mg/kg, with or without 0.7% levorotatory lactic acid. The animals were survived for different periods of time up to one month and no obvious toxic side effects were observed. TEM was used to evaluate the viability of the ascites found in the peritoneal fluid of the mice two weeks post treatment. Cells exhibited characteristic cytostatic morphology consistent with interruption of cell cycling. Therefore we concluded that malachite green and brilliant green have the ability to resolve Ehrlich ascites without significant toxic side effects in this A2G mouse model.

B. Materials and Methods

A lot of 50 A2G mice with a bodyweight of approximately 25 g each were infected with Ehrlich ascites 48 hours prior to treatment. Treatment consisted of one intraperitoneal injection of 1.0 cm³ brilliant green 1:20,000 solution (2 mg per kilogram) or 1.0 cm³ of malachite green 1:10,000 (4 mg per kilogram), with 0.7% levorotatory lactic acid.

Intraperitoneal liquid samples were then extracted at different time periods after the treatment. These samples were taken for electron microscope study of the remaining cells, which confirmed complete inactivation of the malignant cells. Further experimentation was then performed using inactivated Ehrlich ascites in-vitro. In brief, fresh Ehrlich ascites were taken from donors and put into culture. Equal amounts of solution: 1:10,000 brilliant green or 1:5,000 malachite green with 0.7% lactic acid levorotatory was added to the cultures. These mixtures were incubated for 10 minutes at a temperature of 37° C. and stirred. The samples were then centrifuged and the collected ascites were injected intraperitoneally in 5 cm³ doses to each of the A2G mice.

Intraperitoneal liquid was extracted and studied under transmission electron microscopy at 1, 3, 24 and 48 hours after cell transplant. All the animals survived the chronic period and never developed malignant ascites.

C. Electron Microscope Technique

A 3.0% glutaraldehyde solution was added to ascitic liquid extracted from the peritoneal cavity of the mice and stored at room temperature for 15 to 20 minutes. The samples were then centrifuged for 5 min at 1,000 rpm and the supernatant was removed. The ascitic cells were then fixed in a 2.5% glutaraldehyde solution in 0.15 M phosphate buffer, pH 7.2 at a temperature of 40° C. for one hour. The material was then fixed again in 1.0% osmic acid solution 0.15 M phosphate buffer at a temperature of 40° C. for one hour. Acetone dehydration of the solution was performed. The samples were then included into Vestopal W and slicing was performed with an ultramicrotome type LKB-3. Uranyl acetate and lead citrate contrasts were utilized to examine the slides under a Tesla B-613 electron microscope.

D. Experimental Results

In none of the above cases neither the treated mice nor the mice used as cell reservoirs did ever develop ascites.

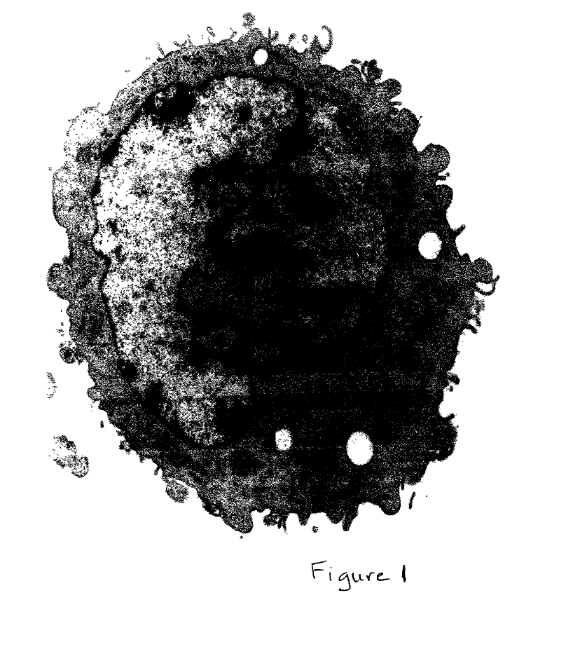

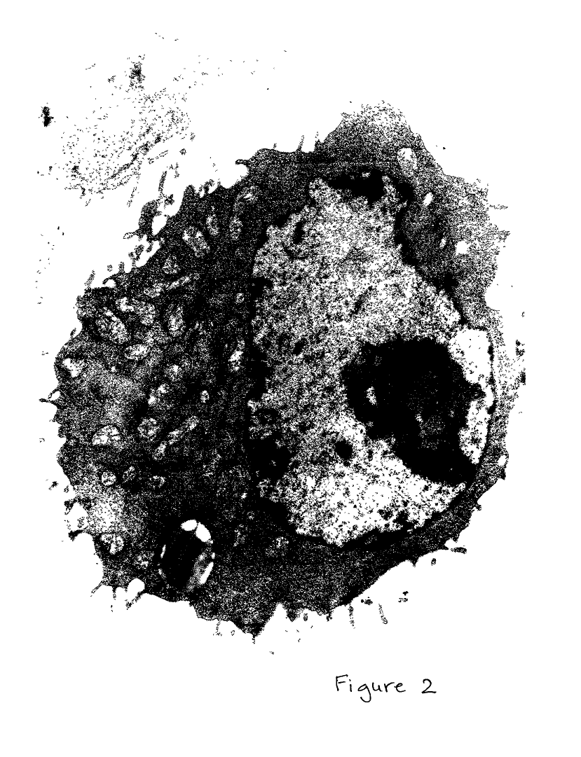

Untreated (control) Ehrlich ascites cells (see FIGS. 1 and 2) are of a spherical shape with a diameter of 8 to 12 microns. The nucleus always has a large nucleolus adherent to the nuclear membrane, indicating an intense synthesis of r-RNA and a high ribosomal transfer in the cytoplasm that is typical to the cancerous cell. In FIG. 2 the nucleolus is expanded. Its fibrillar zone is obvious and the granular zone is typical for the cancerous cell.

After the described treatments, the cytological modifications that were noticed were similar for both triphenylmethane dyes. For that reason in our descriptions we do not mention whether the cells have been treated with malachite green or with brilliant green.

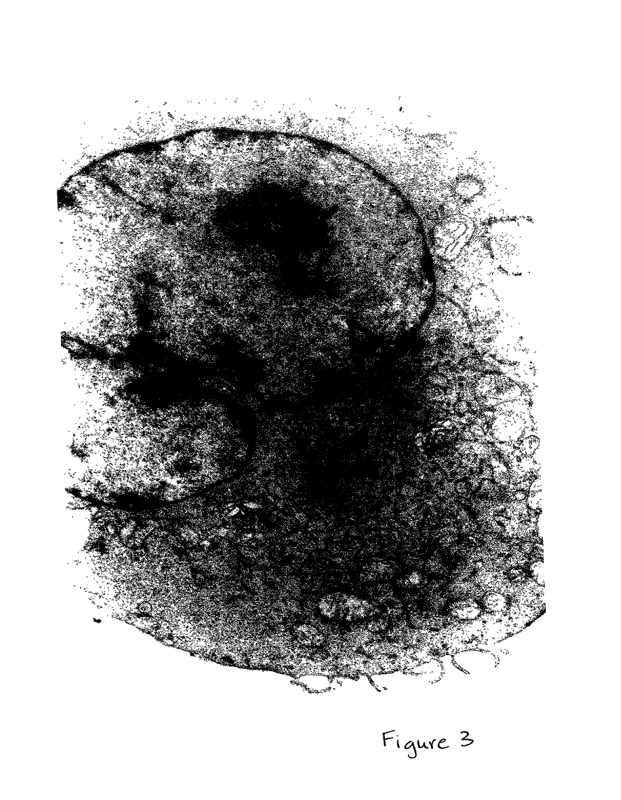

One hour after the treatment (FIG. 3) the following modifications have been noticed as compared to the control cells: intermembranous space of the nucleus is enlarged, the amount of heterochromatin is decreased, and the fragmentation ability of the nucleus is diminished. It is likely that the cells division either becomes rare or completely ceases or there is a maximum of one more cell division possible. The nucleolus does not have a distinct nucleonema. Its central surface looks clear with fibrillar elements and fine granules around. The electron-density of the cytoplasm decreases due to the reduction in the number of ribosomes and mitochondria. Consequently, in some cells the rough endoplasmic reticulum becomes more evident, in others it disappears almost completely. Inside the cytoplasm, a development of large reservoirs or intercytoplasmic vacuoles with numerous and large microvilli lined around them is noted. The Golgi apparatus intensifies its activity and there are numerous electron-dense formations of lysosomes that have a radial disposition around the centriole. The general aspect of the cells shows that their metabolic functions are diminished.

Three hours after the treatment with triphenylmethane dyes the cell aspect is still the same. In addition there are swollen mitochondria with deformed cristae that may be seen in some cells.

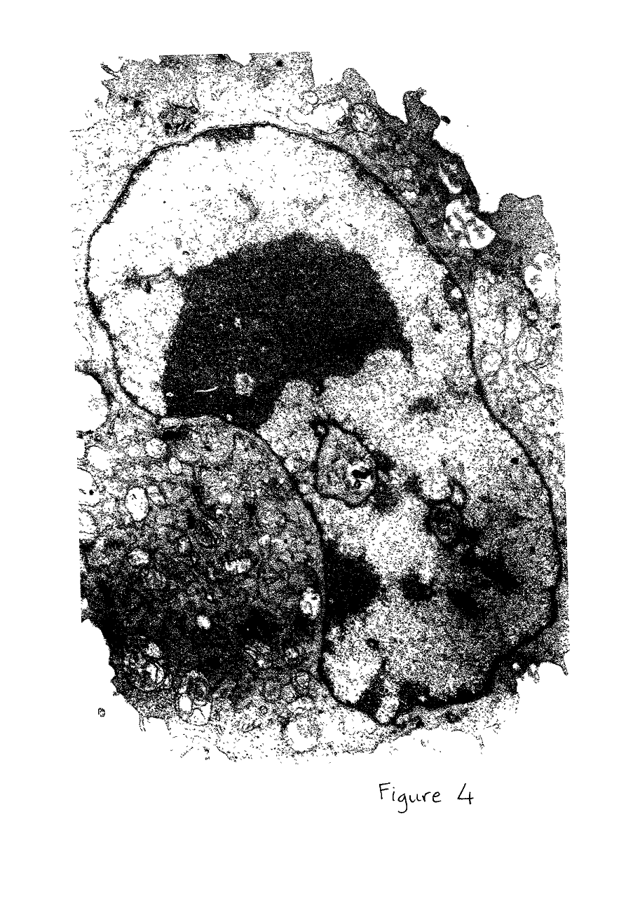

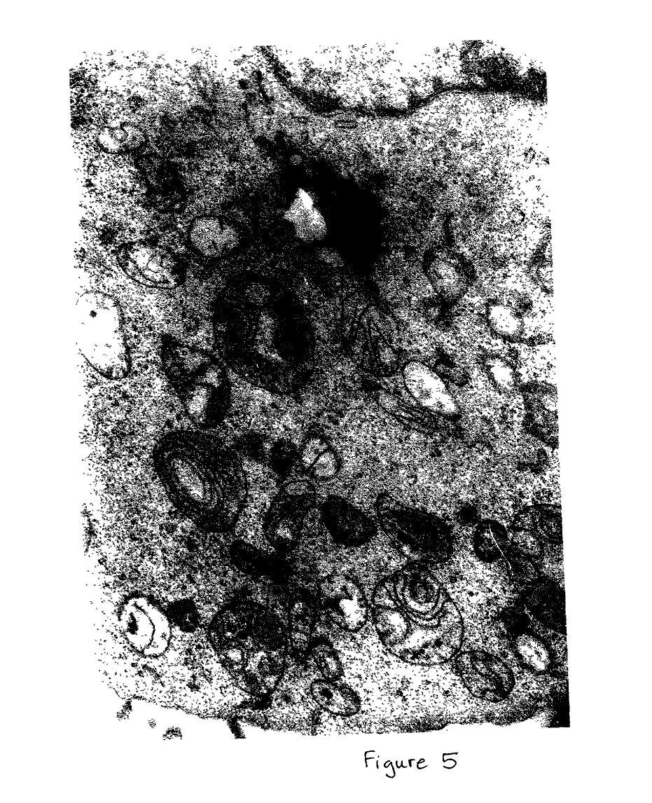

Twenty-four hours after the treatment with triphenylmethane dyes (see FIGS. 4 and 5) the nuclei of the Ehrlich ascites cells are enlarged with less peripheral heterochromatin. The nucleoli are increased and only rarely in contact with the nuclear membrane. This indicates a decrease in the transfer processes between the nucleus and the cytoplasm.

The amount of rough endoplasmic reticulum and the number of ribosomes in the cytoplasm have significantly decreased. Often the membranes of reticulum undergo ribosomal depletion. The mitochondria are less numerous and swollen, their matrix is noticeably less electron-dense and with less cristae (see FIG. 5). These symptoms progress in such a manner that we can find swollen mitochondria with swollen cristae that are transformed into vesicles giving the mitochondria an aspect of multi-vesicular bodies (see FIG. 4).

The intercytoplasmic reservoirs with microvilli are still present. In some cells big vacuoles appear (see FIG. 4). Lipid droplet synthesis is slightly stimulated when the treatment is done with brilliant green, and it is slowed down when the treatment is done with malachite green. In some cells the Golgi apparatus is present in form of multivesicular agglomerations. In other cells, elements of the Golgi apparatus are swollen, which suggests a slowdown of their activity.

There are a large number of cells with formations of lysosomes. Due to their activity many cells undergo a process of pyknosis, which suggests that they are on their way to degeneration or to destruction by antibodies. For the same reason, cells with aberrant forms can also be seen.

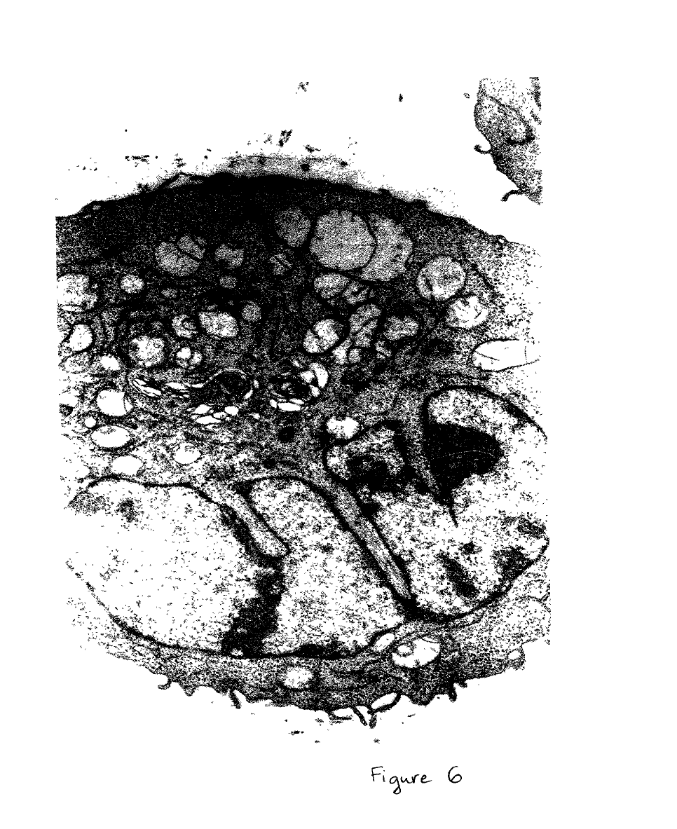

Forty-eight hours after the treatment the appearance of cells is very similar to that after twenty-four hours (see FIGS. 6 to 8). FIG. 6 shows a cell with a fragmented nucleus, swollen mitochondria and lysis of its contents.

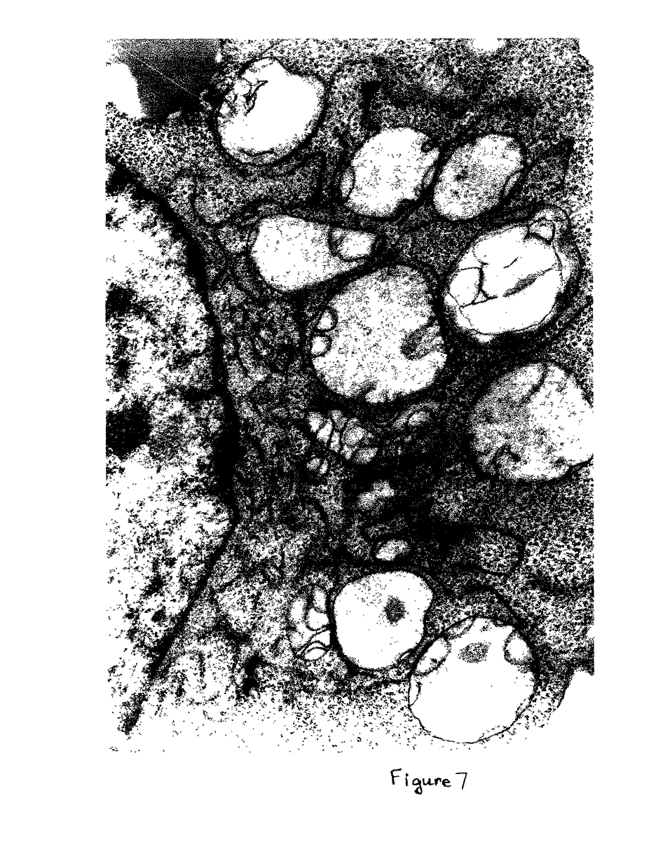

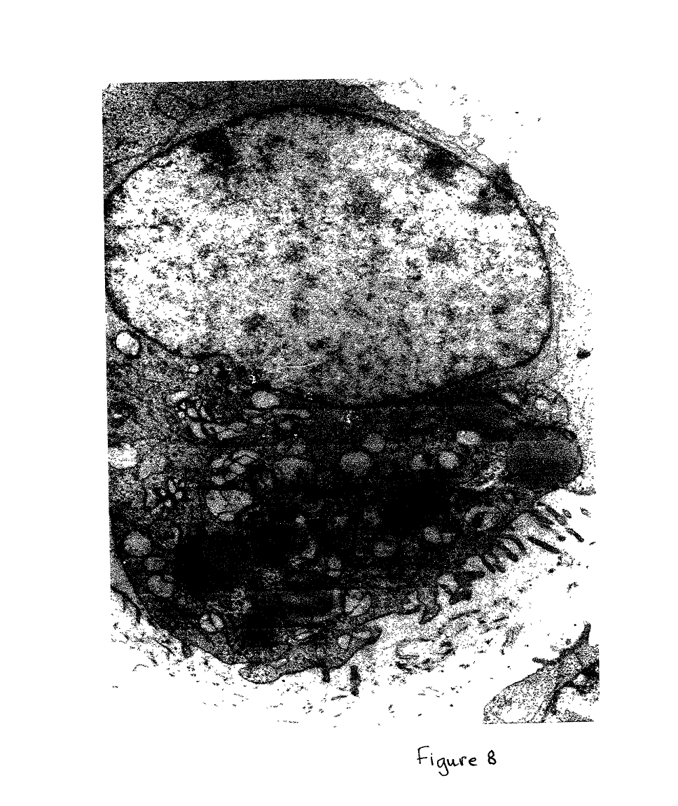

Similar phenomena can be observed in the cytoplasm (FIG. 7). In FIG. 8 the ascitic cell shows an enlarged nucleus without nucleoplasm and there is an accumulation of lipid droplets in the cytoplasm. The mitochondria and the elements of the Golgi apparatus are swollen, inactive and without contents. Many live ascitic cells were extracted two and four weeks after the treatment. These cells have always the aspect of old cells with the nuclei swollen and without chromatin and with a small amount of cytoplasm that has very few organelles. These organelles are swollen and inactive. Very often, these cells have a large number of vacuoles with lipids inside that indicate that they are unable to multiply and in the process of dying.

E. conclusions

Morphologically the effect of malignodepriving inactivation appears evident the first hour after the treatment especially by the permanent nuclear modifications. Other modifications, specific to the phenomenon of senescence, were also observed. In our case, the review of the ultrastructural modifications and the survival for a long time of the blocked (inactivated) cells (we found such blocked cells three and four weeks after the treatments) leads to the conclusion that it is an irreversible phenomenon. This indicates that the cell has lost its malignancy and its possibility of mitosis and therefore it has become compatible with life. The symptoms in the nucleus after the treatment with the antineoplastic compounds presented in this invention having triphenylmethane dyes or isometamidium chloride as the active agent are characteristic and irreversible modifications. The enlargement of the intra-membrane space, a new arrangement of heterochromatin are the ultrastructural alterations typical to the malignodepriving inactivation. A comparison to the ultrastructural modifications caused by cytostatic compounds such as sugar-alcohol derivatives (5) shows that the latter are either lethal or reversible non-specific damages.

The effect of malignodepriving inactivation of malignant cells by treatment with the antineoplastic compounds described in this invention make them suitable for the treatment of cancers.

F. List of Figures

FIGS. 1 and 2: Ehrlich ascites, untreated (control) cells. Irregular shape of nucleus, enlarged nucleoli and a fibrillar zone are evident. A large number of ribosomes can be seen in the cytoplasm. Magnification is 12,000 times.

FIG. 3: Ehrlich ascites cell, one hour after the treatment with brilliant green. The cytoplasm matrix and the nucleoplasm have started to become rarefied. The nucleus and the mitochondria start to become swollen. Magnification is 12,000 times.

FIGS. 4 and 5: Ehrlich ascites cells, twenty-four hours after the treatment. There is a huge increase of the nucleus and nucleolus. The nucleoplasm is rarefied and the membranes are coalescent. The cytoplasm matrix and the mitochondria matrix are rarefied, the mitochondria are swollen and the cristae are myelinized. Magnification is 12,000 times for FIG. 4 and 24,000 times for FIG. 5.

FIGS. 6 and 7: Ehrlich ascites cells, forty-eight hours after the treatment. There is a huge increase of the mitochondria with a complete lysis of its contents. The nucleus is enlarged and fragmented, the nucleoplasm is rarefied and the membranes are coalescent. Magnification 12,000 times for FIG. 6 and 24,000 times for FIG. 7.

FIG. 8: Ehrlich ascites cells, forty-eight hours after the treatment, completely inactivated. The nucleus is enlarged and almost without any nucleoplasm, the cytoplasm has an accumulation of lipidic droplets. Mitochondria and the Golgi apparatus are swollen, inactive and without contents. Magnification 12,000 times.

We claim as our invention:

1. A method of treating cancer by administering a therapeutic composition that inhibits the malignancy of cells.

2. A therapeutic anticancer composition, which comprises a therapeutically effective amount of an anticancer agent, wherein the anticancer agent is a triphenylmethane dye, or pharmaceutically acceptable salts of it.

3. A therapeutic anticancer composition, which comprises a therapeutically effective amount of an anticancer agent, wherein the anticancer agent is isometamidium chloride C28H26C1N7 or 3-amino-8-[3-[3-(aminoiminomethyl)phenyl]-1-triazenyl]-5-ethyl-6-phenyl-,chloride.

4. The composition according to claim 2 used in combination with a vector that is specific to malignant cells.

5. The composition according to claim 3 used in combination with a vector that is specific to malignant cells.

6. The composition with the vector according to claim 4, wherein the vector is lactic acid.

7. The composition with the vector according to claim 5, wherein the vector is lactic acid.

8. A method of treating cancer comprising administering a safe and effective amount of a composition according to claims 2, 4 and 6.

9. A method of treating cancer comprising administering a safe and effective amount of a composition according to claims 3, 5 and 7.

10. A method of treating cancer according to claims 1 to 7 wherein said composition is administered in a liquid form topically, orally, enterically, intravenously, peritoneally, parenterally, by direct injection into the tumor or any other administration route.

11. A method according to claim 10 wherein said liquid dosage form is selected from the group consisting of aqueous solutions, emulsions, suspensions, and suspensions in pharmaceutically acceptable fats or oils.

12. A method of treating cancer according to claims 1 and 2 wherein said triphenylmethane is selected from the group consisting of:
malachite green and salts thereof.

13. A method of treating cancer according to claims 1 and 2 wherein said triphenylmethane is selected from the group consisting of:
brilliant green and salts thereof.
